(19) United States
(12) Patent Application Publication
(10) Pub. No.: US 2015/0307439 A1
(43) Pub. Date: Oct. 29, 2015

(54) FLAVOR AND FRAGRANCE FORMULATION (I)

(71) Applicant: DSM IP ASSETS B.V., Heerlen (NL)

(72) Inventors: Raphael BEUMER, Basel (CH); Johannes TSCHUMI, Basel (CH); Michael GRESSLY, Basel (CH)

(21) Appl. No.: 14/431,892

(22) PCT Filed: Oct. 7, 2013
(86) PCT No.: PCT/EP2013/070836

§ 371 Date: Mar. 27, 2015

(30) Foreign Application Priority Data
Oct. 8, 2012 (EP) 12187637.9

(51) Int. Cl. C07C 69/533; C07C 45/62; C07C 29/44; C07C 67/08; A23L 1/226; C07C 43/178; C07C 33/025; C07C 41/20; C11B 9/00; C07C 69/007; C07C 67/283

(52) U.S. Cl. CPC C07C 69/533; C07C 69/007; C07C 45/62; C07C 29/44; C07C 67/08; C07C 67/283; C07C 43/178; C07C 33/025; C07C 41/20; C11B 9/0019; C11B 9/0015; A23L 1/22657; A23V 2002/00

(57) ABSTRACT

The present invention relates to the use of specific organic compounds as flavor and fragrance material. Furthermore the invention relates to new specific organic compounds and their synthesis, as well to flavor and fragrance formulations comprising at least one of the specific organic compounds.

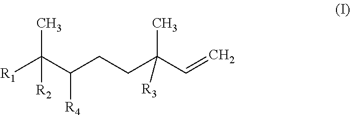

The present invention relates to the use of specific organic compounds as flavor and fragrance material. Furthermore the invention relates to new specific organic compounds, as well as to flavor and fragrance formulations comprising at least one of the specific organic compounds.

In the flavor and fragrance industry there is always a need and demand for compounds that enhance, modify, improve or otherwise positively influence an odor note and therefore give perfumers or other persons the ability to create new fragrances for perfumes, colognes, personal care products, household products or any other products, which comprise flavor and fragrance materials.

Surprisingly it was found that the compounds of formula (I) are very useful as flavor and fragrance materials.

Therefore the present invention is related to the use of a compound of formula (I)

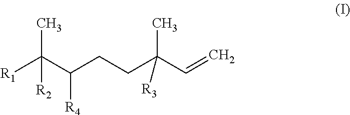

wherein
R₁ signifies —CH₃ (methyl), CH₂CH₃ (ethyl) or —CH₂CH₂CH₃ (n-propyl), and
R₂ signifies —H (hydrogen) or —OCH₃ (methoxy), and
R₃ signifies —OH (hydroxy) or —O(CO)CH₃ (acetyloxy), and
R₄ signifies —H (hydrogen) or —CH₃ (methyl),
   as flavor and fragrance material;
   with the proviso that R¹ is not methyl, if R² signifies hydrogen and R³ signifies hydroxy, and with the further proviso that R¹ is not methyl, if R² and R⁴ signify hydrogen and R³ signifies acetyloxy.

Preferred is the use of at least one compound selected from the group consisting of the compounds of formulae (Ia)-(Ii) and any mixture thereof

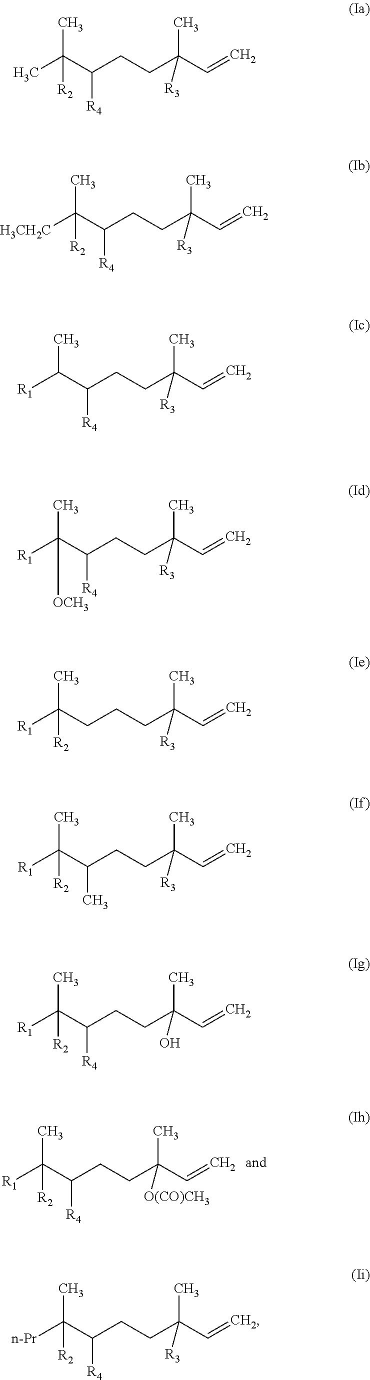

wherein R₁, R₂, R₃ and R₄ have the meanings as defined above,
as flavor and fragrance material.

Among these the use of a compound of formula (Id) or of a compound of formula (Ii) or of any mixture thereof is even more preferred. Most preferred among these is the use of a compound of formula (Id).

More preferred is the use of at least one compound selected from the group consisting of compounds of formulae (II)-(XII) (whereby compounds of formulae (VI) and (X) are especially preferred) and any mixture thereof

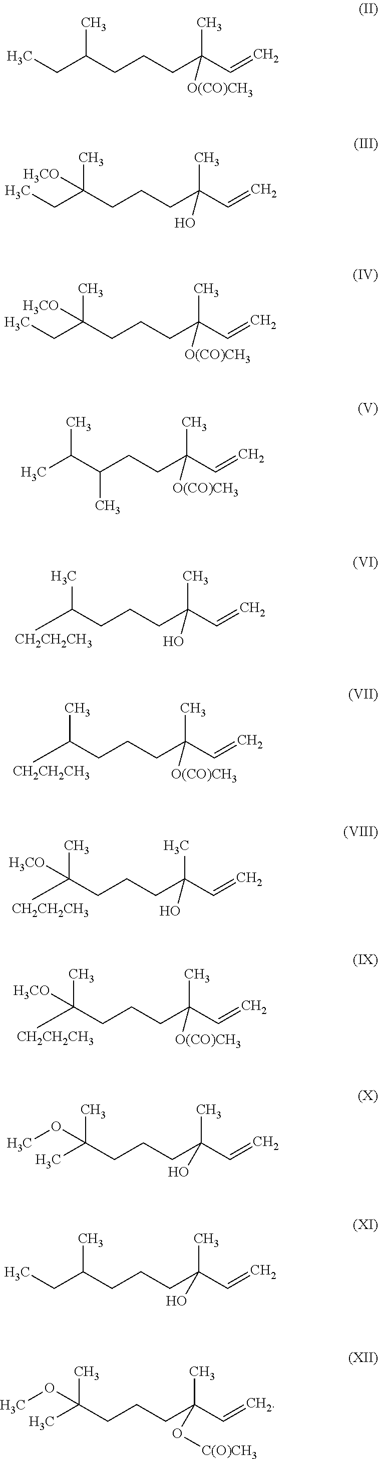

Most preferred is the use of compound (X).

The compounds of formula (I) may be used as such or in combination with other compounds of formula (I) or other compounds which are known as flavor and fragrance material.

Such other compounds which are known as flavor and fragrance material include all known odorant molecules selected from the extensive range of natural products and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in flavor fragrance formulations, for example, carrier materials, and other auxiliary agents commonly used in the art.

The flavor and fragrance material of the present invention is used in a flavor and fragrance formulation.

Such a flavor and fragrance formulation comprises other ingredients.

The flavor and fragrance formulation according to the present invention can be in any form. Usually it is in a solid, gel-like or liquid (or a combination thereof) form. It can also be in an encapsulated form (i.e. a liquid formulation encapsulated by a suitable matrix material).

Therefore the present invention also relates to flavor and fragrance formulations comprising
(i) at least one compound of formula (I)

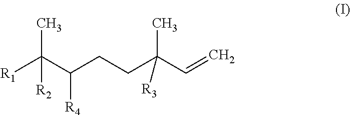

wherein
   R₁ signifies —CH₃, CH₂CH₃ or —CH₂CH₂CH₃, and
   R₂ signifies —H or —OCH₃, and
   R₃ signifies —OH or —O(CO)CH₃, and
   R₄ signifies —H or —CH₃;
with the proviso that R¹ is not methyl, if R² signifies hydrogen and R³ signifies hydroxy, and
with the further proviso that R¹ is not methyl, if R² and R⁴ signify hydrogen and R³ signifies acetyloxy;
and with the preferences as given above.

Preferred are flavor and fragrance formulations comprising at least one compound selected from the group consisting of the compounds of formulae (Ia)-(Ii) and their mixtures

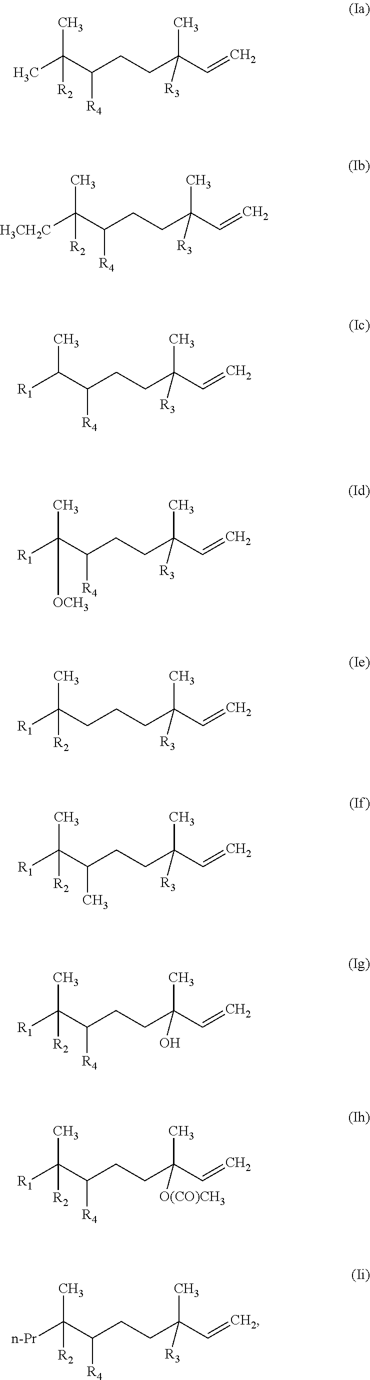

wherein R₁, R₂, R₃ and R₄ have the meanings as defined above, and wherein the compounds of formula (Id) and (Ii) are especially preferred.

More preferred are flavor and fragrance formulations comprising at least one compound selected from the group consisting of the compounds of formulae (II)-(XII) and their mixtures

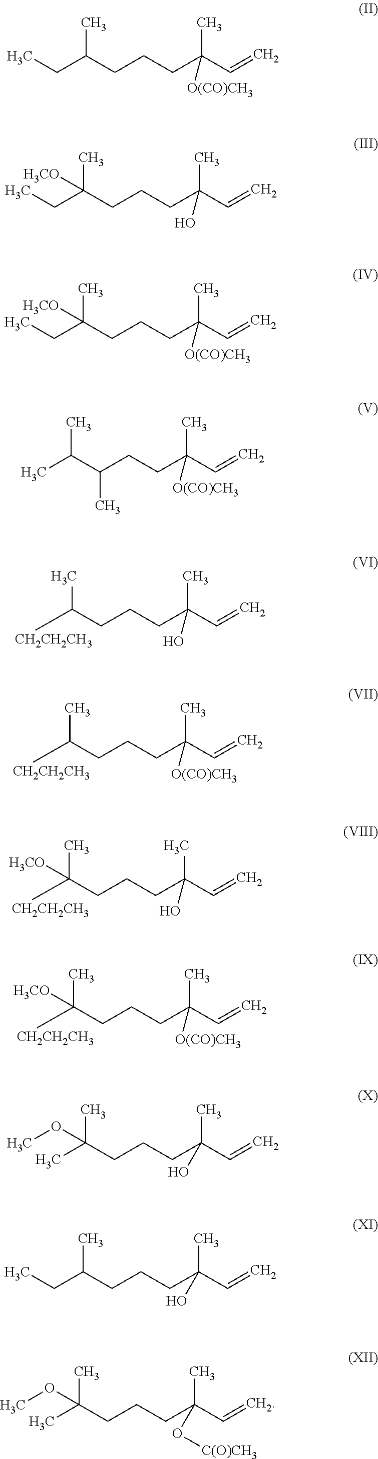

When a compound of formula (I) with the preferences as given above is used in a flavor and fragrance formulation, then the amount thereof is in the range of 0.0001-10 weight-% (wt-%), related to the total weight of the flavor and fragrance formulation. Preferably is an amount in the range of 0.01-5 wt-%, based on the total weight of the flavor and fragrance formulation.

Therefore the present invention relates to liquid flavor and fragrance formulations comprising
(i) 0.0001-10 wt-% (preferably 0.01-5 wt-%), related to the total weight of the flavor and fragrance formulation, of at least one compound of formula (I) with the preferences as given above.

The flavor and fragrance formulations according to the present invention can comprise further ingredients (=auxiliary compounds), such as any further perfuming compounds solvents, adjuvants, thickeners, surface active agents, pigments, extenders, rheology modifiers, dyestuffs, antioxidants, fillers and the like.

Many flavor and fragrance formulations are in a liquid form (like a perfume, cologne, etc.). Therefore, for such liquid formulation a (diluent) solvent is present. Such common diluents are i.e. dipropyleneglycol, isopropyl myristate, triethylcitrate and alcohols (such as ethanol).

Further examples of fine perfumery are Eau de perfume, Eau de Toilette, Eau de Cologne and Splash Cologne. Fine perfumery products are commonly based on an alcoholic solution as diluent. However fine perfumery products using an oil or wax as diluent are also included within the meaning of this invention. The compounds can be employed in widely varying amounts, depending upon the specific application and on the nature and quantity of other odourant ingredients.

When used in a (fine) perfume, the amount of the compound of formula (I) with the preferences as given above is usually between 0.01-10 wt-%, based on the total weight of the (fine) perfume.

However, these values and ranges are given only by way of example, since the experienced perfumer may also achieve effects or may create novel accords with lower or higher concentrations.

Furthermore the present invention relates to liquid flavor and fragrance formulations comprising
(i) at least one compound of formula (I) with the preferences as given above, and
(ii) at least one diluent chosen from the group consisting of dipropyleneglycol, isopropylmyristate, triethylcitrate and alcohols (such as ethanol), and optionally
(iii) at least one auxiliary compound selected from the group consisting of perfuming compounds solvents, adjuvants, thickeners, surface active agents, pigments, extenders, rheology modifiers, dyestuffs, antioxidants and fillers.

Furthermore the present invention relates to solid flavor and fragrance formulations comprising
(i) at least one compound of formula (I) with the preferences as given above and
(ii) at least one auxiliary compound selected from the group consisting of perfuming compounds solvents, adjuvants, thickeners, surface active agents, pigments, extenders, rheology modifiers, dyestuffs, antioxidants and fillers.

The compounds of formula (I) with the preferences as given above may be used in a broad range of flavor and fragrance formulations, e.g. in any field of fine and functional perfumery, such as perfumes, air care products, household products, laundry products, body care products and cosmetics.

The compounds as described hereinabove may be employed in a flavor and fragrance formulation simply by directly mixing at least one compound of formula (I) with the preferences as given above, a mixture thereof, or a fragrance composition with the other ingredients used in the final product, or they may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, or they may be chemically bonded to substrates, which are adapted to release the fragrance molecule upon application of an external stimulus such as light, enzyme, or the like, and then mixed with the other ingredients used in the final product.

Thus, the invention additionally provides a method of manufacturing a flavor and fragrance formulation, comprising the incorporation of a compound of formula (I) with the preferences as given above, as a fragrance ingredient, either by directly admixing the compound to the other ingredients used in the final product or by admixing a fragrance composition comprising a compound of formula (I) with the preferences as given above, which may then be mixed with the other ingredients used in the final product, using conventional techniques and methods.

Through the addition of an olfactory acceptable amount of a compound of the present invention as hereinabove described, or a mixture thereof, the odor notes of a consumer product base will be improved, enhanced or modified.

Thus, the invention furthermore provides a method for improving, enhancing or modifying a consumer product (=final product) base by means of the addition thereto of an olfactory acceptable amount of a compound of formula (I) with the preferences as given above, or a mixture thereof.

In the context of the present invention the olfactory effective amount is to be understood as the amount of the at least one compound of formula (I) with the preferences as given above in a flavor and fragrance formulation will contribute to its particular olfactory characteristics, but the olfactory effect of the flavor and fragrance formulation will be the sum of the effects of each of the perfumes or fragrance ingredients. Thus the compounds of the invention can be used to alter the aroma characteristics of the flavor and fragrance formulation, or by modifying the olfactory reaction contributed by another ingredient in the composition. The amount will vary depending on many factors including other ingredients, their relative amounts and the effect that is desired.

As used herein, “consumer product (=final product)” means a composition for use as a consumer product to fulfill specific actions, such as cleaning, softening, and caring or the like. Examples of such products include fine perfumery, e.g. perfume and eau de toilette; fabric care, household products and personal care products such as laundry care detergents, rinse conditioner, personal cleansing composition, detergent for dishwasher, surface cleaner; laundry products, e.g. softener, bleach, detergent; body care products, e.g. shampoo, shower gel; air care products and cosmetics, e.g. deodorant, vanishing creme. This list of products is given by way of illustration and is not to be regarded as being in any way limiting.

The compounds of formula (I) may be prepared using methods known to the person skilled in the art of organic synthesis. They may be especially prepared according to a process starting from a compound of formula (XIII)

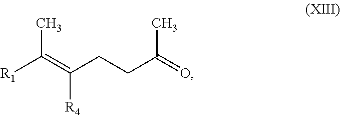

wherein R₁ signifies methyl, ethyl or n-propyl, and R₄ signifies hydrogen or methyl, and whereby
ia) the compound of formula (I) is hydrogenated at the C═C double bond to a compound of formula (XIV) with R₁ as defined above and with R₅=—H, if a compound of formula I with R₂=hydrogen is manufactured, or

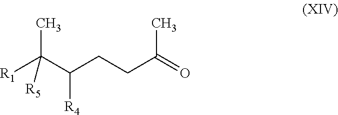

ib) the compound of formula (I) is methoxylated to a compound of formula (XIV) with R₁ as defined above and with R₅=—OCH₃, if a compound of formula I with R₂=methoxy is manufactured, and
ii) the compound of formula (XIV) obtained in step ia) with R₅=—H or in step ib) with R₅=—OCH₃ is then ethinylated to a compound of formula (XV), and

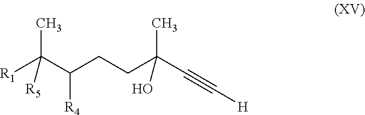

iii) only in case when R₃=—O(CO)CH₃ the compound of formula (XV) is acylated to a compound of formula (XVI) with R₁ and R₅ as defined above, and

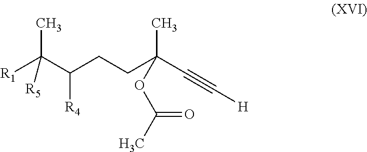

(iv) the compound of formula (XV) obtained in step ii) or the compound of formula (XVI) obtained in step iii) are hydrogenated to a compound of formula (I).

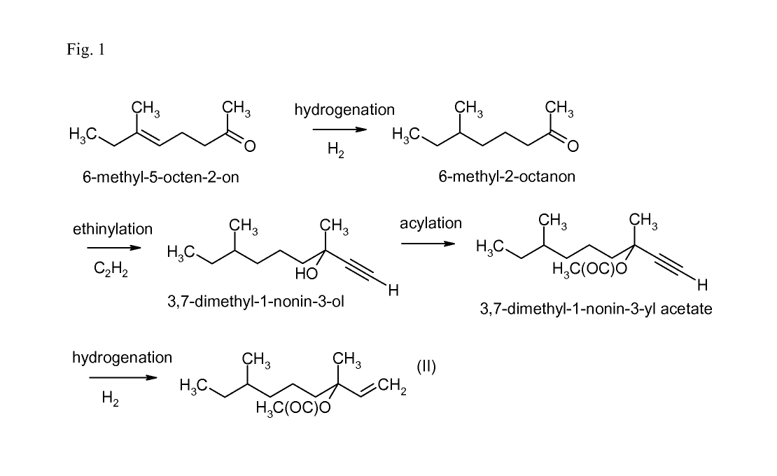

Such a process is also part of the present invention, as well as a process for the manufacture of a compound of formula (II) to (IX) and (XII) as shown in FIGS. 1-6.

Furthermore the present invention relates to the following compounds of formulae (II)-(IX) and (XII) which are novel compounds:

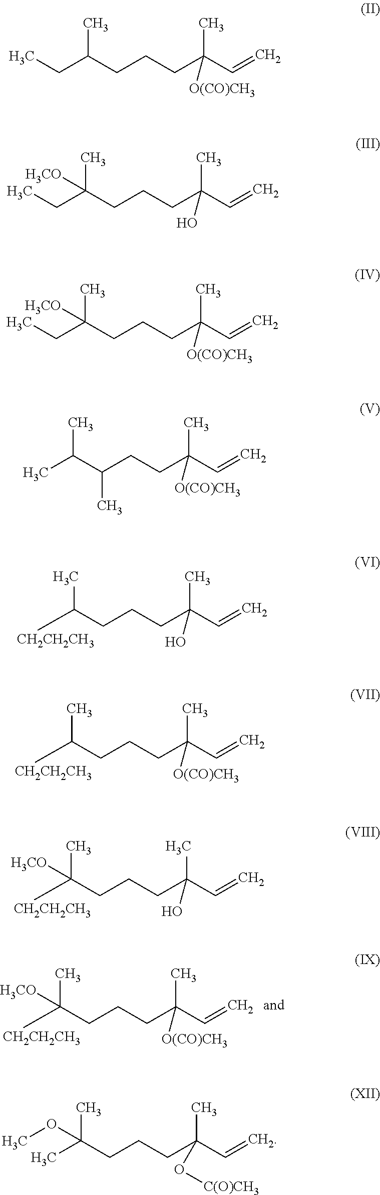

These novel compounds may be produced (manufactured, synthesized) as disclosed below. The present invention is also directed to their synthesis.

For example compound of formula (II) can be manufactured as shown in FIG. 1 starting from 6-methyl-5-octen-2-on, which is selectively hydrogenated to 6-methyl-2-octanon. 6-Methyl-2-octanon is ethinylated to 3,7-dimethyl-1-nonin-3-ol. After transformation of the hydroxy group to an acetyloxy group the thus obtained 3,7-dimethyl-1-nonin-3-yl acetate is hydrogenated in the presence of a Lindlar catalyst to the compound of formula (II).

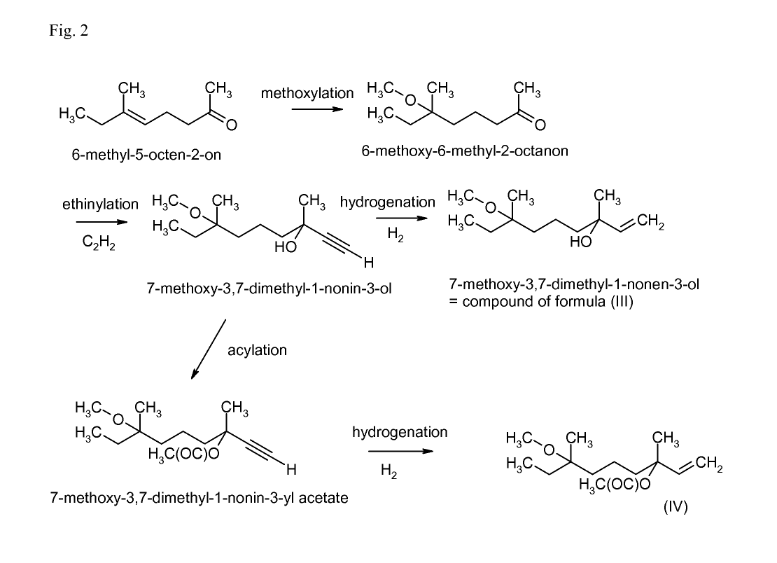

Compound of formula (III) may also be prepared starting from 6-methyl-5-octen-2-on to which first methanol is added. After the resulting 6-methoxy-6-methyl-2-octanon has been ethinylated to 7-methoxy-3,7-dimethyl-1-nonin-3-ol the thus obtained compound is hydrogenated in the presence of a Lindlar catalyst to the compound of formula III. This reaction sequence is shown in FIG. 2. If, however, the hydroxy group of 7-methoxy-3,7-dimethyl-1-nonin-3-ol is first transformed in an aceteloxy group and then the triple bond is hydrogenated, this leads to the compound of formula (IV).

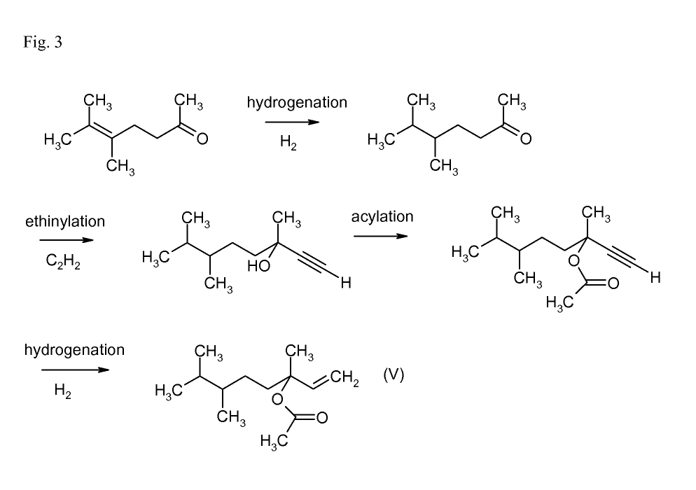

Compound of formula (V) may be manufactured starting from 5,6-dimethyl-5-hepten-2-one which is first selectively hydrogenated at the double bond, then ethinylated and after “protecting” the hydroxy group as acetyloxy group selectively hydrogenated to the compound of formula (V) in the presence of a Lindlar catalyst as shown in FIG. 3.

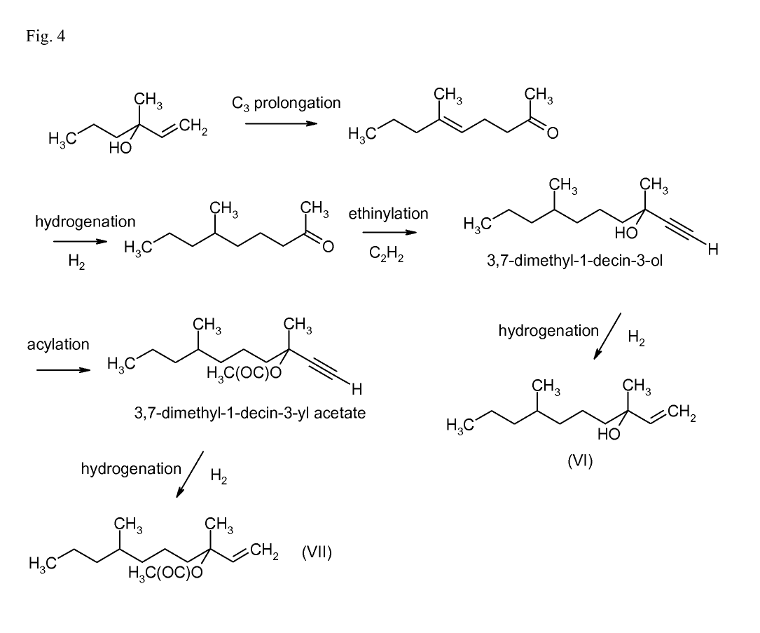

The compounds of formula (VI) and (VII) may be manufactured starting from 3-methyl-1-hexen-3-ol as shown in FIG. 4. 3-methyl-1-hexen-3-ol is reacted with IPM (isopropenyl methyl ether) to 6-methyl-5-nonen-2-on. After selective hydrogenation of the double bond an ethinylation takes place leading to 3,7-dimethyl-1-decin-3-ol. Hydrogenation of the triple to the double bond leads to the compound of formula (VI), whereas when an acylation is performed before the hydrogenation the compound of formula (VII) is obtained.

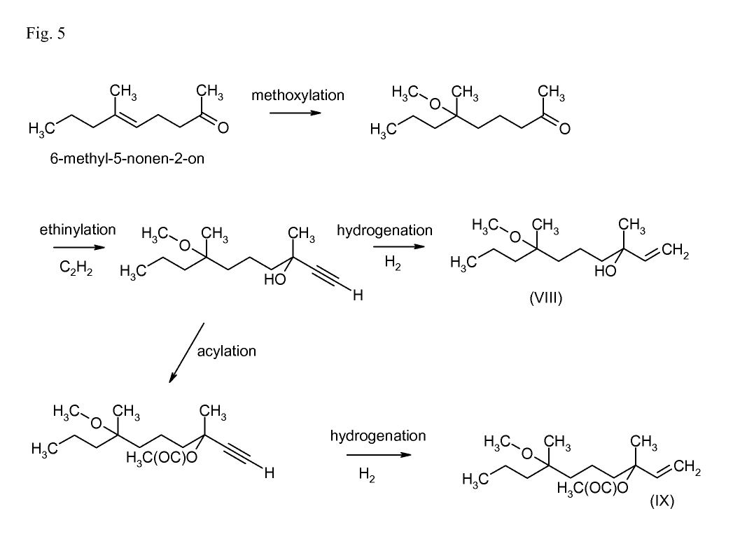

6-methyl-5-nonen-2-on can also be the starting material for compounds (VIII) and (IX) as shown in FIG. 5: After addition of MeOH (methanol) an ethinylation is carried out. Hydrogenation of the triple to the double bond leads to compound of formula (VIII), whereas when an acylation is performed before the hydrogenation the compound of formula (IX) is obtained.

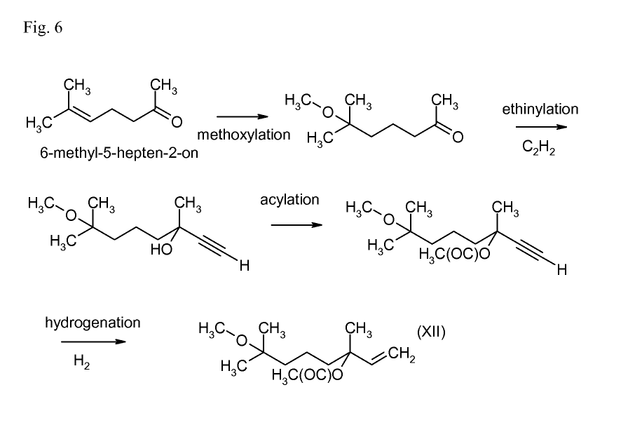

The manufacture of the compound of formula (XIII) is outlined in FIG. 6: First methanol (MeOH) is added to 6-methyl-5-hepten-2-on. Afterwards an ethinylation takes place. After having transformed the hydroxy in an acetyloxy group the triple bond is hydrogenated to a double bond, thus leading to the compound of formula (XIII).

The ethinylations may be carried out as e.g. disclosed in EP-A 1 432 667 and EP-A 1 532 092.

The invention is now further illustrated in the following non-limiting examples.

### EXAMPLES

All compounds were evaluated by a panel of four persons for their intensity whereby a range of 1 to 10 was used (1=very low intensity; 10=very high intensity). Furthermore these four persons also described the odor of the compounds. The tenancy was evaluated by one person after 3, 6, 8, 24, 48, 72 and 96 hours. For such evaluations a piece of paper was immersed in each single liquid compound as such.

### Example 1

### Manufacture of the Compound of Formula II and its Olfactory Properties

### a) Manufacture of 6-methyl-2-octanon by hydrogenation of 6-methyl-5-octen-2-on

900.0 g of 6-methyl-5-octen-2-on and 1.00 g of 5% Pd on carbon (Pd/C) are put in an autoclave under nitrogen and heated to a temperature of 60° C. under stirring. Then the nitrogen is exchanged by hydrogen and put to an absolute pressure of 2 bar. After the calculated amount of hydrogen has been consumed the reaction mixture is filtered and distilled (5 mbar, 85° C.) to obtain 6-methyl-2-octanon.

### b) Manufacture of 3,7-dimethyl-1-nonin-3-ol by ethinylation of 6-methyl-2-octanon

1020.0 g of 6-methyl-2-octanon are put in an autoclave under nitrogen and cooled down to a temperature of 15° C. 1920.0 g of ammonia (NH₃) are added. The reaction mixture is cooled again to 15° C. Then acetylene (C₂H₂) is added. The reaction mixture is cooled again to 15° C. Then 19.80 g of a 40 weight-% aqueous potassium hydroxide (KOH) solution are added continuously. After the end of the reaction the reaction mixture is neutralized with acetic acid, extracted with water and the solvent removed. The resulting raw product is then distilled to obtain 3,7-dimethyl-1-nonin-3-ol.

### c) Manufacture of 3,7-dimethyl-1-nonin-3-yl acetate

440.23 g of 3,7-dimethyl-1-nonin-3-ol and 0.45 g of p-toluene sulfonic acid in water are mixed and heated up to a temperature of 40° C. 345.40 g of acetic acid anhydride are added within 2 hours. After ca. 20 hours the reaction mixture is cooled down and distilled to obtain 3,7-dimethyl-1-nonin-3-yl acetate.

### d) Manufacture of compound of formula (II) by hydrogenation of 3,7-dimethyl-1-nonin-3-yl acetate

366.95 g of 3,7-dimethyl-1-nonin-3-yl acetate, 7.5 g of Lindlar catalyst (5% Pd+3.5% Pb on CaCO₃), 0.04 g of ethylenedithiodiethanol and 0.02 g of zinc acetate are put in an autoclave and heated under nitrogen to a temperature of 45° C. Nitrogen is exchanged by hydrogen (H₂) and the reaction mixture put at an absolute pressure of 2 bar. After the calculated amount of hydrogen has been consumed the reaction mixture is filtered and distilled (1 mbar, 120° C.) to obtain 3,7-dimethyl-1-nonene-3-yl acetate (=compound of formula II).

### e) Olfactory Properties

Odor description: pleasantly good; fresh green wood; nut note; cosmetic powder; sweet woodruff; lemonade powder.

### Intensity: 5.

Tenancy: 3-6 hours.

### Example 2

### Manufacture of the Compound of Formula III and its Olfactory Properties

### a) Manufacture of 6-methoxy-6-methyl-2-octanon by methoxylation of 6-methyl-5-octen-2-on

6600.0 g of 6-methyl-5-octen-2-on and 9390.0 g of methanol are mixed in a reactor and cooled to 0° C. 4462.8 g of H₂SO₄ (96 weight-%) are added within 40 minutes by keeping the temperature. The reaction mixture is then heated up to 30° C. After 5 hours the reaction mixture is poured on ice water and extracted with methyl tert-butyl ether. The combined methyl tert-butyl ether phases are washed with 20 weight-% aqueous Na₂CO₃ solution and concentrated NaCl solution. The solvent is removed from the organic phase and the residue distilled (5 mbar; 125° C.) to yield 6-methoxy-6-methyl-2-octanon.

### b) Manufacture of 7-methoxy-3,7-dimethyl-1-nonin-3-ol by ethinylation of 6-methoxy-6-methyl-2-octanon

1265.0 g of 6-methoxy-6-methyl-2-octanon are put in an autoclave under nitrogen and cooled down to a temperature of 15° C. 3226.0 g of ammonia (NH₃) are added. The reaction mixture is cooled again to 15° C. Then acetylene (C₂H₂) is added. The reaction mixture is cooled again to 15° C. Then 33.0 g of a 40 weight-% aqueous potassium hydroxide (KOH) solution are added continuously. After the end of the reaction the reaction mixture is neutralized with acetic acid, extracted with water and the solvent removed. The resulting raw product is then distilled to obtain 7-methoxy-3,7-dimethyl-1-nonin-3-ol.

### c) Manufacture of the compound of formula (III) by hydrogenation of 7-methoxy-3,7-dimethyl-1-nonin-3-ol

313.88 g of 7-methoxy-3,7-dimethyl-1-nonin-3-ol, 3.0 g of Lindlar catalyst (5% Pd+3.5% Pb on CaCO₃) and 0.03 g of ethylenedithiodiethanol are put in an autoclave and heated under nitrogen to a temperature of 55° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 135° C.) to obtain 7-methoxy-3,7-dimethyl-1-nonen-3-ol (=compound of formula III).

### d) Olfactory Properties

Odor description: forest soil; pine; natural resin; fresh wood; a trace of eucalyptus.

### Intensity: 5.5.

Tenancy: 48-72 hours.

### Example 3

### Manufacture of the Compound of Formula IV and its Olfactory Properties

7-Methoxy-3,7-dimethyl-1-nonin-3-ol is prepared as described above under example 2a-c.

### a) Manufacture of 7-methoxy-3,7-dimethyl-1-nonin-3-yl acetate

707.0 g of 7-methoxy-3,7-dimethyl-1-nonin-3-ol and 0.57 g of p-toluene sulfonic acid in water are mixed and heated up to a temperature of 60° C. 437.1 g of acetic acid anhydride are added within 2 hours. After ca. 20 hours the reaction mixture is cooled down and distilled to obtain 7-methoxy-3,7-dimethyl-1-nonin-3-yl acetate.

### b) Manufacture of the compound of formula (IV) by hydrogenation of 7-methoxy-3,7-dimethyl-1-nonin-3-yl acetate

250.0 g of 7-methoxy-3,7-dimethyl-1-nonin-3-yl acetate, 2.0 g of Lindlar catalyst (5% Pd+3.5% Pb on CaCO₃) and 0.03 g of ethylenedithiodiethanol are put in an autoclave and heated under nitrogen to a temperature of 40° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 140° C.) to obtain 7-methoxy-3,7-dimethyl-1-nonen-3-yl acetate (=compound of formula IV).

### c) Olfactory Properties

Odor description: thuya; coriander; spices; fresh.

### Intensity: 4.5.

Tenancy: 24-48 hours.

### Example 4

### Manufacture of the Compound of Formula V and its Olfactory Properties

### a) Manufacture of 5,6-dimethyl-2-heptanon by hydrogenation of 5,6-dimethyl-5-hepten-2-on

1400.0 g of 5,6-dimethyl-5-hepten-2-on and 3.3 g of 5% Pd/C are put in an autoclave and heated under nitrogen to a temperature of 60° C. Nitrogen is exchanged by hydrogen (H₂) and the reaction mixture put at an absolute pressure of 2 bar. After the calculated amount of hydrogen has been consumed the reaction mixture is filtered and distilled (5 mbar, 85° C.) to obtain 5,6-dimethyl-2-heptanon.

### b) Manufacture of 3,6,7-trimethyl-1-octin-3-ol by ethinylation of 5,6-dimethyl-2-heptanon

1272.0 g of 5,6-dimethyl-2-heptanon are put in an autoclave under nitrogen and cooled down to a temperature of 15° C. 2557.0 g of ammonia (NH₃) are added. The reaction mixture is cooled again to 15° C. Then acetylene (C₂H₂) is added. The reaction mixture is cooled again to 15° C. Then 26.4 g of a 40 weight-% aqueous potassium hydroxide (KOH) solution are added continuously. After the end of the reaction the reaction mixture is neutralized with acetic acid, extracted with water and the solvent removed. The resulting raw product is then distilled to obtain 3,6,7-trimethyl-1-octin-3-ol.

### c) Manufacture of 3,6,7-trimethyl-1-octin-3-yl acetate

757.4 g of 3,6,7-trimethyl-1-octin-3-ol and 0.73 g of p-toluene sulfonic acid in water are mixed and heated up to a temperature of 40° C. 555.1 g of acetic acid anhydride are added within 2 hours. After ca. 20 hours the reaction mixture is cooled down and distilled to obtain 3,6,7-trimethyl-1-octin-3-yl acetate.

### d) Manufacture of the compound of formula (V) by hydrogenation of 3,6,7-trimethyl-1-octin-3-yl acetate

667.0 g of 3,6,7-trimethyl-1-octin-3-yl acetate, 11.0 g of Lindlar catalyst (5% Pd+3.5% Pb on CaCO₃) and 0.03 g of ethylenedithiodiethanol are put in an autoclave and heated under nitrogen to a temperature of 45° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 115° C.) to obtain 3,6,7-trimethyl-1-octen-3-yl acetate (=compound of formula V).

### e) Olfactory Properties

Odor description: dry leaves; fresh washed clothes.

### Intensity: 2.

Tenancy: 3-6 hours.

### Example 5

### Manufacture of the Compound of Formula VI and its Olfactory Properties

### a) Manufacture of 6-methyl-2-nonanon by hydrogenation of 6-methyl-5-nonen-2-on

900.0 g of 6-methyl-5-nonen-2-on and 1.6 g of 5% Pd/C are put in an autoclave and heated under nitrogen to a temperature of 60° C. Nitrogen is exchanged by hydrogen (H₂) and the reaction mixture put at an absolute pressure of 2 bar. After the calculated amount of hydrogen has been consumed the reaction mixture is filtered and distilled (10 mbar, 120° C.) to obtain 6-methyl-2-nonanon.

### b) Manufacture of 3,7-dimethyl-1-decin-3-ol by ethinylation of 6-methyl-2-nonanon

760.0 g of 6-methyl-2-nonanon are put in an autoclave under nitrogen and cooled down to a temperature of 15° C. 1305.0 g of ammonia (NH₃) are added. The reaction mixture is cooled again to 15° C. Then acetylene (C₂H₂) is added. The reaction mixture is cooled again to 15° C. Then 14.0 g of a 40 weight-% aqueous potassium hydroxide (KOH) solution are added continuously. After the end of the reaction the reaction mixture is neutralized with acetic acid, extracted with water and the solvent removed. The resulting raw product is then distilled to obtain 3,7-dimethyl-1-decin-3-ol.

### c) Manufacture of the compound of formula (VI) by hydrogenation of 3,7-dimethyl-1-decin-3-ol

220.0 g of 3,7-dimethyl-1-decin-3-ol, 0.25 g of Lindlar catalyst (5% Pd+3.5% Pb on CaCO₃) and 0.04 g of ethylenedithiodiethanol are put in an autoclave and heated under nitrogen to a temperature of 45° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 120° C.) to obtain 3,7-dimethyl-1-decen-3-ol (=compound of formula VI).

### d) Olfactory Properties

Odor description: cleaning agent; wet sand; clean; fresh; citron.

### Intensity: 4.5

Tenancy: 8-24 hours.

### Example 6

### Manufacture of the Compound of Formula VII and its Olfactory Properties

3,7-dimethyl-1-decin-3-ol is prepared as described above in example 5 a) to b).

### a) Manufacture of 3,7-dimethyl-1-decin-3-yl acetate

472.0 g of 3,7-dimethyl-1-decin-3-ol and 0.42 g of p-toluene sulfonic acid in water are mixed and heated up to a temperature of 40° C. 317.4 g of acetic acid anhydride are added within 2 hours. After ca. 20 hours the reaction mixture is cooled down and distilled to obtain 3,7-dimethyl-1-decin-3-yl acetate.

### b) Manufacture of the compound of formula (VII) by hydrogenation of 3,7-dimethyl-1-decin-3-yl acetate

310.0 g of 3,7-dimethyl-1-decin-3-yl acetate, 2.4 g of Lindlar catalyst (5% Pd+3.5% Pb on CaCO₃), 0.01 g of ethylenedithiodiethanol and 0.11 g of zinc acetate are put in an autoclave and heated under nitrogen to a temperature of 45° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 140° C.) to obtain 3,7-dimethyl-1-decen-3-yl acetate (=compound of formula VII).

### c) Olfactory Properties

Odor description: vinegar; green; herb; grass.

### Intensity: 3.5.

Tenancy: 8-24 hours.

### Example 7

### Manufacture of the Compound of Formula VIII and its Olfactory Properties

### a) Manufacture of 6-methoxy-6-methyl-nonan-2-on by methoxylation of 6-methyl-5-nonen-2-on

6404.0 g of 6-methyl-5-nonen-2-on, 10400.0 g of methanol and 2050.0 g of Amberlyst 15 wet are mixed in a reactor and heated up to 80° C. After 15 hours the reaction mixture is cooled and reduced in volume by distilling. The remaining residue is then distilled (2 mbar; 145° C.) to give 6-methoxy-6-methyl-nonan-2-on.

### b) Manufacture of 7-methoxy-3,7-dimethyl-1-decin-3-ol by ethinylation of 6-methoxy-6-methyl-2-nonanon

1020.0 g of 6-methoxy-6-methyl-2-nonanon are put in an autoclave under nitrogen and cooled down to a temperature of 15° C. 1931.0 g of ammonia (NH₃) are added. The reaction mixture is cooled again to 15° C. Then acetylene (C₂H₂) is added. The reaction mixture is cooled again to 15° C. Then 19.8 g of a 40 weight-% aqueous potassium hydroxide (KOH) solution are added continuously. After the end of the reaction the reaction mixture is neutralized with acetic acid, extracted and the solvent removed. The resulting raw product is then distilled to obtain 7-methoxy-3,7-dimethyl-1-decin-3-ol.

### c) Manufacture of the compound of formula (VIII) by hydrogenation of 7-methoxy-3,7-dimethyl-1-decin-3-ol

220.0 g of 7-methoxy-3,7-dimethyl-1-decin-3-ol, 0.20 g of Lindlar catalyst (5% Pd+3.5% Pb on CaCO₃) and 0.04 g of ethylenedithiodiethanol are put in an autoclave and heated under nitrogen to a temperature of 45° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 140° C.) to obtain 7-methoxy-3,7-dimethyl-1-decen-3-ol (=compound of formula VIII).

### d) Olfactory Properties

Odor description: rubber boat.

### Intensity: 3.5.

Tenancy: 72-96 hours.

### Example 8

### Manufacture of the Compound of Formula IX and its Olfactory Properties

The manufacture of 7-methoxy-3,7-dimethyl-1-decin-3-ol is already described in example 7 a) and b).

### a) Manufacture of 7-methoxy-3,7-dimethyl-1-decin-3-yl acetate by acylation of 7-methoxy-3,7-dimethyl-1-decin-3-ol

430.0 g of 7-methoxy-3,7-dimethyl-1-decin-3-ol and 0.32 g of p-toluene sulfonic acid in water are mixed and heated up to a temperature of 40° C. 248.3 g of acetic acid anhydride are added within 2 hours. After ca. 20 hours the reaction mixture is cooled down and distilled to obtain 7-methoxy-3,7-dimethyl-1-decin-3-yl acetate.

### b) Manufacture of the compound of formula (IX) by hydrogenation of 7-methoxy-3,7-dimethyl-1-decin-3-yl acetate

250.0 g of 7-methoxy-3,7-dimethyl-1-decin-3-yl acetate, 1.5 g of Lindlar catalyst (5% Pd+3.5% Pb on CaCO₃), 0.01 g of ethylenedithiodiethanol and 0.07 g of zinc acetate are put in an autoclave and heated under nitrogen to a temperature of 40° C. Nitrogen is exchanged by hydrogen (H₂) and the reaction mixture put at an absolute pressure of 2 bar. After the calculated amount of hydrogen has been consumed the reaction mixture is filtered and distilled (1 mbar, 150° C.) to obtain 7-methoxy-3,7-dimethyl-1-decen-3-yl acetate (=compound of formula IX).

### c) Olfactory Properties

Odor description: vinegar; fresh; fresh green wood.

### Intensity: 2.

Tenancy: 72-96 hours.

### Example 9

### Olfactory Properties of the Compound of Formula X

Odor description: pleasantly fresh; slightly camphor like; tiger balsam; forest; pine; coniferous wood.

### Intensity: 2.5.

Tenancy: 8-24 hours.

### Example 10

### Olfactory Properties of the Compound of Formula XI

Odor description: fresh; good; angenehm; citrus fruits; baby powder; creme.

### Intensity: 5.

Tenancy: 3-6 hours.

### Example 11

### Manufacture and Olfactory Properties of the Compound of Formula XII

### a) Manufacture of 6-methoxy-6-methyl-2-heptanon by methoxylation of 6-methyl-5-hepten-2-on

6000.0 g of 6-methyl-5-hepten-2-on and 9480.0 g of methanol are mixed in a reactor and cooled to 0° C. 4507.8 g of H₂SO₄ (96 weight-%) are added within 40 minutes by keeping the temperature. The reaction mixture is then heated up to 30° C. After 5 hours the reaction mixture is poured on ice water and extracted with methyl tert-butyl ether. The combined methyl tert-butyl ether phases are washed with 20 weight-% aqueous Na₂CO₃ solution and concentrated NaCl solution. The solvent is removed from the organic phase and the residue distilled (10 mbar; 130° C.) to yield 6-methoxy-6-methyl-heptan-2-on.

### b) Manufacture of 7-methoxy-3,7-dimethyl-1-octin-3-ol by ethinylation of 6-methoxy-6-methyl-2-heptanon

2566.4 g of 6-methoxy-6-methyl-2-heptanon are put in an autoclave under nitrogen and cooled down to a temperature of 15° C. 5221.0 g of ammonia (NH₃) are added. The reaction mixture is cooled again to 15° C. Then acetylene (C₂H₂) is added. The reaction mixture is cooled again to 15° C. Then 52.8 g of a 40 weight-% aqueous potassium hydroxide (KOH) solution are added continuously. After the end of the reaction the reaction mixture is neutralized with acetic acid, extracted and the solvent removed. The resulting raw product is then distilled to obtain 7-methoxy-3,7-dimethyl-1-octin-3-ol.

### c) Manufacture of 7-methoxy-3,7-dimethyl-1-octin-3-yl acetate

560.0 g of 7-methoxy-3,7-dimethyl-1-octin-3-ol and 0.49 g of p-toluene sulfonic acid in water are mixed and heated up to a temperature of 40° C. 372.5 g of acetic acid anhydride are added within 2 hours. After ca. 20 hours the reaction mixture is cooled down and distilled to obtain 7-methoxy-3,7-dimethyl-1-octin-3-yl acetate.

### d) Manufacture of the compound of formula (XII) by hydrogenation of 7-methoxy-3,7-dimethyl-1-octin-3-yl acetate

656.0 g of 7-methoxy-3,7-dimethyl-1-octin-3-yl acetate, 11.0 g of Lindlar catalyst (5% Pd+3.5% Pb on CaCO₃) and 0.14 g of ethylenedithiodiethanol are put in an autoclave and heated under nitrogen to a temperature of 40° C. Nitrogen is exchanged by hydrogen (H₂) and the reaction mixture put at an absolute pressure of 2 bar. After the calculated amount of hydrogen has been consumed the reaction mixture is filtered and distilled (2 mbar, 135° C.) to obtain 7-methoxy-3,7-dimethyl-1-octen-3-yl acetate (=compound of formula XII).

### e) Olfactory Properties

Odor description: fine; warm; sweet; gummy bears (fruit gums/jellybabies).

### Intensity: 3.

Tenancy: 8-24 hours.

1. Use of a compound of formula (I) 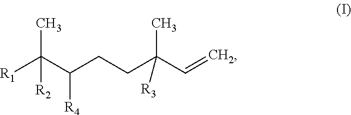
wherein
R₁ signifies —CH₃, —CH₂CH₃ or —CH₂CH₂CH₃, and
R₂ signifies —H or —OCH₃, and
R₃ signifies —OH or —O(CO)CH₃, and
R₄ signifies —H or —CH₃;
with the proviso that R¹ is not methyl, if R² signifies hydrogen and R³ signifies hydroxyl; and
with the further proviso that R¹ is not methyl, if R² and R⁴ signify hydrogen and R³ signifies acetyloxy;
as flavor and fragrance material.

2. Use according to claim 1, wherein at least one compound of formulae (Ia)-(Ii) 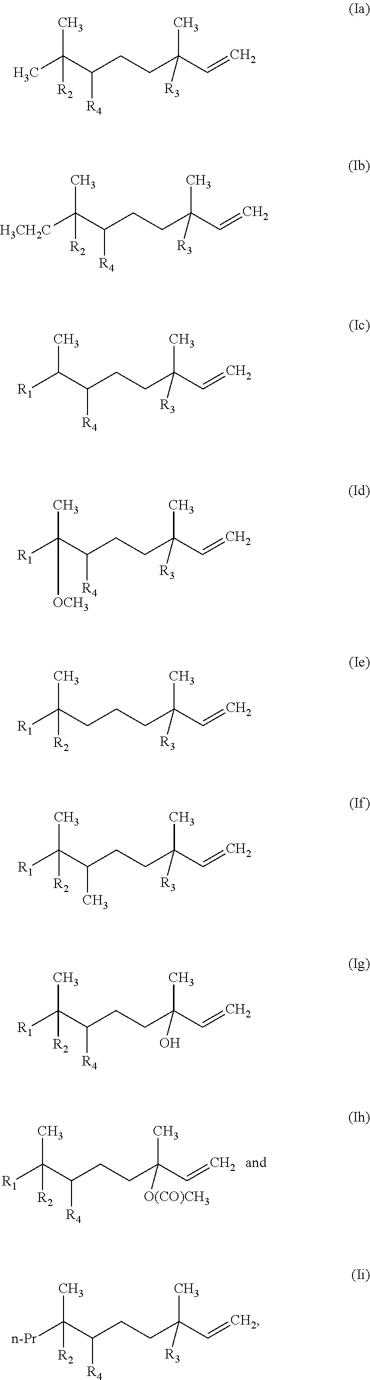
wherein R₁, R₂, R₃ and R₄ have the meanings as defined in claim 1,
is used.

3. A flavor and fragrance formulation comprising
(i) at least one compound of formula (I) 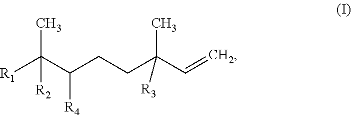
wherein
R₁ signifies —CH₃, —CH₂CH₃ or —CH₂CH₂CH₃, and
R₂ signifies —H or —OCH₃, and
R₃ signifies —OH or —O(CO)CH₃, and
R₄ signifies —H or —CH₃;
with the proviso that R¹ is not methyl, if R² signifies hydrogen and R³ signifies hydroxyl; and
with the further proviso that R¹ is not methyl, if R² and R⁴ signify hydrogen and R³ signifies acetyloxy.

4. Flavor and fragrance formulation according to claim 3 comprising 0.0001-10 wt-%, related to the total weight of the flavor and fragrance formulation, of at least one compound of formula (I).

5. Flavor and fragrance formulation according to claim 3, wherein the flavor and fragrance formulation is solid, gel-like or liquid.

6. Flavor and fragrance formulation according to claim 3, wherein the flavor and fragrance formulation is a perfume, air care product, household product, laundry product, body care product or cosmetic product.

7. A method of improving, enhancing or modifying a flavor and fragrance formulation by means of addition thereto an olfactory acceptable amount of at least one compound of formula (I) 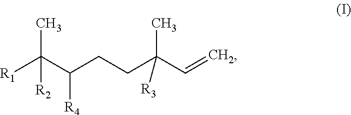
wherein
R₁ signifies —CH₃, —CH₂CH₃ or —CH₂CH₂CH₃, and
R₂ signifies —H or —OCH₃, and
R₃ signifies —OH or —O(CO)CH₃, and
R₄ signifies —H or —CH₃;
with the proviso that R¹ is not methyl, if R² signifies hydrogen and R³ signifies hydroxyl;
and with the further proviso that R¹ is not methyl, if R² and R⁴ signify hydrogen and R³ signifies acetyloxy.

8. A compound of formula (II)-(IX) and (XII)

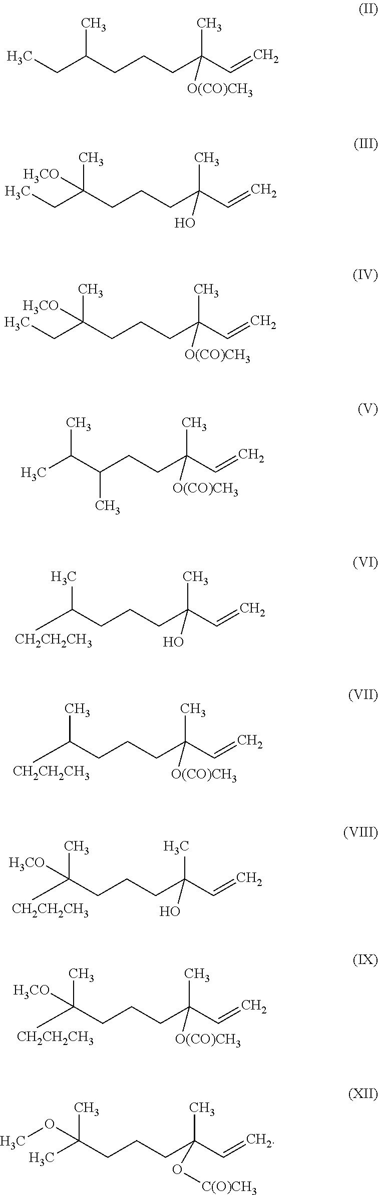

9. A process for the manufacture of a compound of formula (I) 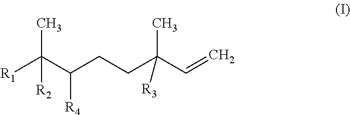
as defined in claim 1
starting from a compound of formula (XIII) 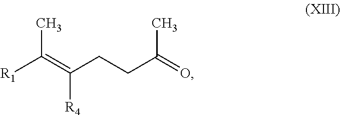
wherein R₁ signifies methyl, ethyl or n-propyl, and R₄ signifies hydrogen or methyl, and whereby
ia) the compound of formula (I) is hydrogenated at the C═C double bond to a compound of formula (XIV) with R₁ as defined above and with R₅=—H, if a compound of formula I with R₂=hydrogen is manufactured, or 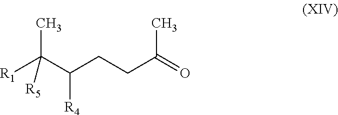
ib) the compound of formula (I) is methoxylated to a compound of formula (XIV) with R₁ as defined above and with R₅=—OCH₃, if a compound of formula I with R₂=methoxy is manufactured, and
ii) the compound of formula (XIV) obtained in step ia) with R₅=—H or in step ib) with R₅=—OCH₃ is then ethinylated to a compound of formula (XV), and 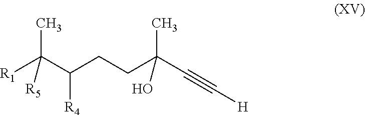
iii) only in case when R₃=—O(CO)CH₃ the compound of formula (XV) is acylated to a compound of formula (XVI) with R₁ and R₅ as defined above, 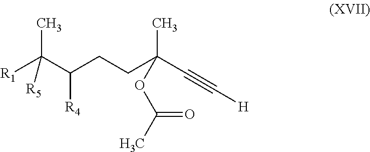
and
(iv) the compound of formula (XV) obtained in step ii) or the compound of formula (XVI) obtained in step iii) are hydrogenated to a compound of formula (I).

10. A process for the manufacture of a compound of formula (II) to (IX) and (XII) as shown in FIGS. 1-6.
